SEQUENCE LISTING (1 sequence)

SEQ ID NO: 1 - protein, 527 aa, Artificial sequence
  Synthetic W253R/R275S tPA
SYQVICRDEKTQMIYQQHQSWLRPVLRSNRVEYCWCNSGRAQCHSVPVKSCSEPRCFNGGTCQQALYFSDFVCQCPEGFAGKCCEIDTRATCYEDQGISYRGTWSTAESGAECTNWNSSALAQKPYSGRRPDAIRLGLGNHNYCRNPDRDSKPWCYVFKAGKYSSEFCSTPACSEGNSDCYFGNGSAYRGTHSLTESGASCLPWNSMILIGKVYTAQNPSAQALGLGKHNYCRNPDGDAKPWCHVLKNRRLTREYCDVPSCSTCGLRQYSQPQFSIKGGLFADIASHPWQAAIFAKHRRSPGERFLCGGILISSCWILSAAHCFQERFPPHHLTVILGRTYRVVPGEEEQKFEVEKYIVHKEFDDDTYDNDIALLQLKSDSSRCAQESSVVRTVCLPPADLQLPDWTECELSGYGKHEALSPFYSERLKEAHVRLYPSSRCTSQHLLNRTVTDNMLCAGDTRSGGPQANLHDACQGDSGGPLVCLNDGRMTLVGIISWGLGCGQKDVPGVYTKVTNYLDWIRDNMRP

(19) United States
(12) Patent Application Publication
(10) Pub. No.: US 2023/0263870 A1
(43) Pub. Date: Aug. 24, 2023

(54) SLOW RELEASE PLASMINOGEN ACTIVATOR FORMULATION FOR USE IN THE TREATMENT OF THROMBOTIC OR HAEMORRHAGIC DISEASE

(71) Applicant: OP2LYSIS, Boulogne-Billancourt (FR)

(72) Inventors: Jérôme PARCQ, Hermanville sur mer (FR); Christophe GAUDIN, Saint Cloud (FR); Estelle LOUET, Caen (FR); Marie-Julie GUICHARD, Paris (FR)

(21) Appl. No.: 17/998,432

(22) PCT Filed: May 11, 2021
(86) PCT No.: PCT/EP2021/062400

§ 371 Date: Nov. 10, 2022

(30) Foreign Application Priority Data
May 11, 2020 (EP) 20305462.2

(51) Int. Cl. A61K 38/49; A61K 9/51; A61K 47/10; A61P 7/02

(52) U.S. Cl. CPC A61K 38/49; A61K 9/5192; A61K 47/10; A61P 7/02; C12Y 304/21068

(57) ABSTRACT

The present invention relates to slow release plasminogen activator composition. The present invention also relates to the therapeutic use of said composition, in particular in thrombotic or haemorrhagic disease.

### FIELD OF THE INVENTION

The present invention relates to slow release plasminogen activator composition. The present invention also relates to the therapeutic use of said composition, in particular in thrombotic or haemorrhagic disease.

### BACKGROUND OF THE INVENTION

Intracerebral haemorrhage (ICH) is the most severe form of stroke that affects up to 5 million inhabitants per year worldwide (Krishnamurthi, R V et al. 2015. Neuroepidemiology ; 45(3):

190-202; Feigin, V L et al. 2014. Lancet; 383(9913): 245-54). Most of them die or remain seriously disabled. Case fatality rate at 30 days is in the range 40-50% while only 20% are expected to be functionally independent at 6 months (van Asch, et al. 2010, The Lancet. Neurology 9(2): 167-762010; Sacco, et al. 2009. Stroke 40(2): 394-99). At the Kaplan-Meier analysis, the 10-year survival rate was 24.1%. Current guidelines recommendations in EU and USA (Steiner et al. 2014. International Journal of Stroke: Official Journal of the International Stroke Society; 9(7): 840-55 ; Hemphill, et al. 2015. Stroke; 46 (7): 2032-60) are to admit stroke patients in specialized stroke Intensive Care Units (ICU) with the following objectives: (1) to reverse any coagulation abnormality (2) to maintain normoglycemia (3) to control blood pressure and (4) to prevent thrombo-embolic events and decubitus complications. No specific treatment has yet been approved for intracerebral haemorrhage (ICH).

Most instances of ICH occur when small arteries rupture with subsequent leaking of blood into the brain, leading to the fast constitution of a solid and massive intracerebral haematoma. The mass-effect of the haematoma destroys surrounding brain tissues and compresses remote brain regions, leading to high fatality or severe motor and cognitive disability, the primary injury. Inflammatory processes triggered as soon as the haematoma formation, the secondary injury, result in the formation of a perihaematomal oedema (PHE). While the PHE volume may appear as a valuable marker in clinical studies to predict patient outcome (Schlunk F, et al. 2015. Translational Stroke Research; 6(4): 257-63.; Urday, et al. 2015. Nat. Rev. Neurol.; 11(2): 111-22), a clear priority is to reduce the volume of the haematoma, either by stopping bleeding within the first hours or by evacuating haematoma within the first days to limit the secondary injury.

Therapeutic targets to reduce the burden of ICH were investigated as extended application of existing drugs with the objective to stop early the bleeding (e.g. use of recombinant factor VIIa, FAST trial (Mayer, et al. 2008. The New England Journal of Medicine; 358(20): 2127-37) or to limit late inflammatory process with neuroprotective strategies (e.g. use of an iron chelator, deferoxamine Hi-Def trial (Yeatts, et al. 2013. Neurocritical Care; 19(2): 257-66). Nevertheless, none of these strategies showed benefits for ICH patients.

Another strategy is to extract the haematoma to reduce the mass-effect of the haematoma and subsequent secondary injury. While the first results using standard surgery were not positive regarding the primary endpoint (Mendelow, et al. 2013. Lancet; 382(9890): 397-408.), new ways to access the haematoma were investigated to reduce the deleterious effects of the surgery. Minimally invasive surgery with thrombolysis in intracerebral haemorrhage evacuation (MisTIE trial) showed drastically reduced side effect (Hanley et al. 2019. Lancet, 383(10175):1021-1032). The investigators showed that MisTIE strategy reduced ICH mortality and, more interestingly, they also demonstrated a strong relationship between the residual haematoma volume and the recovery of ICH patients (Awad, et al. 2019. Neurosurgery, 84(6):1157-1168). Thus, the MisTIE strategy may be the right solution for ICH patients. Nevertheless, the right thrombolytic agent should be used to ensure an increased probability of low disability.

Tissue plasminogen activator (tPA) is the only medicine (recombinant Thrombolytic agent) approved for the treatment of Ischemic Stroke. This 527 amino-acids glycoprotein is composed of five domains from the N-terminal end to the C-terminal end: finger domain, EGF-like domain, two kringle domains and the proteolytic domain (Van Zonneveld, et al. 1986. Journal of Cellular Biochemistry; 32(3): 169-78). Due to its ability to activate plasminogen into plasmin at low concentration, tPA favours thrombus resolution (Hoylaerts et al. 1982). Recombinant tPA (rtPA) has been proposed as a pharmaceutical option for the treatment of myocardial infarction, acute ischemic stroke (AIS) and pulmonary embolism (Quinn, et al. 2008. Expert Review of Neurotherapeutics; 8(2): 181-92).

During the last 20 years, rtPA has been deeply evaluated for the treatment of haemorrhagic stroke as a thrombolytic agent, intraventricular haemorrhage in the CLEAR trials and intracerebral haemorrhage in the MisTIE trials. The MisTIE program proposes the injection of recombinant tPA (rtPA, alteplase) in the intracerebral haematoma, using a thin catheter to reach the target zone, and by which the liquefied blood following thrombolytic action of rtPA is drained. MisTIE phase 3 trial did not reach the primary endpoint (reduction of the mRS score in the MisTIE treated group) (Hanley et al. 2019. Lancet, 383(10175):1021-1032). While rtPA is a strong thrombolytic agent, it suffers from three limitations in the treatment of acute cerebral conditions: firstly, it does not allow fast, efficient and safe haematoma evacuation. Indeed, rtPA treatment allows only approx. 69% haematoma reduction after 72 hrs and up to 9 injections. Secondly, it triggers rebleeding (+25% vs control group) (Hanley et al. 2019. Lancet, 383(10175):1021-1032). Thirdly, rtPA has been shown to be a neuromodulator of the glutamatergic neurotransmission leading to potential side effects that limit its beneficial therapeutic effect in a model of ICH in pigs (Rohde et al. 2002. Journal of Neurosurgery; 97(4): 954-62; Thiex, et al. 2007. Journal of Neurosurgery; 106(2): 314-20.; Keric et al. 2012. Translational Stroke Research; 3 (Suppl 1): 88-93).

In the past two decades, several experimental studies focused on extending the rtPA therapeutic time window in order to improve its thrombolytic efficacy. Nevertheless, most drugs have failed in clinical trials. Nanocarriers were designed and fabricated to prevent deactivation of rtPA during circulation and allow rapid release of rtPA near blood clot region (Chung et al. 2008, Biomaterials; 29(2):228-237).

However, protein and protease loading in a carrier is always an issue. Indeed, processes applied in the formulation of drugs induce both chemical and physical stress on the formulated compounds. This may result in a degradation and loss of activity of sensible therapeutics such as proteins and peptides.

Initiatives to encapsulate and deliver rtPA over an extended duration were conducted, but limitation regarding either the efficiency of encapsulation (less than 75%) or the concentration of the protein encapsulated (0.1 mg/ml) were highlighted (Chung et al. 2008, Biomaterials; 29(2):228-237; Wang, et al. 2009, J Biomed Mater Res; 91A:753-61; Zhou et al. 2014; ACS applied materials & interfaces; 6:5566-76; Sivaraman et al. 2016; Mater Sci eng C Mater Biol Appl. 59:145-156).

Thus, it remains a need to develop new formulations that limit the side effects of plasminogen activator while improving its efficacy on thrombolysis.

### SUMMARY OF THE INVENTION

The inventors surprisingly showed that sustained release of plasminogen activator in the core of a haematoma allows a better thrombolytic efficacy (reduction of the clot weight) than repeated injections of the same quantity of the gold standard tissue plasminogen activator (rtPA).

Thus, they investigated a methodology to prepare plasminogen activator compositions from high concentration stock solution to allow slow release over a few hours in order to reduce the number of injection as well as the time needed to achieve a substantial reduction of the haematoma.

To do so, a new brain-compatible formulation has been developed with plasminogen activator nanoparticles and thermoreversible polymer in order to allow high concentration loading of plasminogen activator and a release time of less than 24 hours while maintaining plasminogen activator efficacy.

The present disclosure relates to a composition comprising a thermoreversible polymer and a nanoparticle comprising a plasminogen activator.

In a particular embodiment, said nanoparticle comprises a poloxamer, preferably selected from the group consisting of: 188, 338 and 237, more preferably poloxamer 188.

In another particular embodiment, said thermoreversible polymer is a poloxamer, preferably a poloxamer 407. Said poloxamer 407 is preferably at a concentration between 15 to 25% (w/v), more preferably between 17 and 23% (w/v), again more preferably 17% (w/v).

In a particular embodiment, said plasminogen activator is selected from the group consisting of Alteplase, Tenecteplase, Pamiteplase, Monteplase, lanoteplase, reteplase, desmoteplase, urokinase or streptokinase. In another particular embodiment, said plasminogen activator is a mutated plasminogen activator, preferably W253R/R275S mutant plasminogen activator, more preferably W253R/R275S mutant plasminogen activator consisting of the sequence SEQ ID NO: 1.

In another aspect, the present disclosure relates to said composition for use as medicament, preferably for treating thrombotic or haemorrhagic disease. Said thrombotic or haemorrhagic disease is selected from the group consisting of: thrombotic or embolic ischemia, artery or vein occlusions, deep haematoma, cerebral haemorrhages or haematoma and ocular haemorrhages or haematoma, preferably from intra-parenchymatous haemorrhages or haematoma, intra-ventricular haemorrhages or haematoma, cerebral subarachnoid haemorrhages or haematoma, age related macular degeneration, central retinal occlusion, vitreous haemorrhages, any deep haematoma traumatic or not, any post-surgical haematoma including cerebral haematoma or following intervention for cancer, more preferably cerebral haemorrhages or haematoma such as intra-parenchymatous, intra-ventricular and subarachnoid haemorrhages or haematoma.

In a further aspect, the present disclosure also relates to a method of preparing a composition comprising a thermoreversible polymer and a plasminogen activator-poloxamer nanoparticle, said method comprising the steps of: i) preparing an aqueous solution comprising a plasminogen activator and a poloxamer, ii) contacting the obtained solution with a protein precipitation solvent in a sufficient amount to precipitate plasminogen activator in combination with a poloxamer to form a plasminogen activator-poloxamer nanoparticle, iii) adding said nanoparticle in a thermoreversible polymer.

In a last aspect, the present disclosure relates to a nanoparticle comprising a plasminogen activator in combination with a poloxamer, preferably poloxamer 188.

### FIGURE LEGENDS

FIG. 1: Amidolytic and fibrinolytic characterization of OptPA vs rtPA. A) Mean amidolytic activity of rtPA and OptPA (W253R/R275S tPA) toward spectrozyme® rtPA chromogenic substrate, measured in the range of concentration from 0 to 200 ng per well and in absence of fibrin (n=4). B) Detail of the amidolytic activity of OptPA and rtPA at the different concentrations tested. Amidolytic activity is expressed as the maximal velocity (optical density per second per μg) (10 concentrations tested, n=4 per concentration); C) Maximal turbidity assessed by optical density at 405 nanometers for each concentration of OptPA and rtPA tested (n=5); D) Fibrinolytic activity measured as the time to reach 50 per cent lysis with OptPA or rtPA in a whole plasma clot lysis assay (pool of human plasma, n=5). * means p<0.05; ****, p<0.0001; ns=non-significant; Open square, light grey=OptPA; plain square, dark grey=rtPA.

FIG. 2: Amidolytic characterization of OptPA₁₅₁ vs rtPA alteplase. A) Mean amidolytic activity of alteplase and OptPA₁₅₁ toward spectrozyme® rtPA chromogenic substrate, measured in the range of concentration from 0 to 200 ng per well and in absence of fibrin. B) Detail of the amidolytic activity of OptPA₁₅₁ and alteplase at the different concentrations tested. C) Comparison of mean amidolytic activity of OptPA (obtained from a minipool, prior to clone isolation, FIG. 1A) and OptPA₁₅₁ (derived from the selected research cell bank, FIG. 2A) toward spectrozyme® rtPA chromogenic substrate, measured in the range of concentration from 0 to 200 ng per well and in absence of fibrin. ** means p<0.01; ****, p<0.0001; Open circle=OptPA₁₅₁; open square=rtPA; plain circle=OptPA & plain square=rtPA from FIG. 1A.

FIG. 3: Plasminogen activation assay using OptPA₁₅₁ vs alteplase (rtPA). Plasminogen activator, plasminogen and pNAPEP, a specific substrate of plasmin, were incubated in a Tris 50 mM-NaCl 150 mM pH=8.0 buffer. Absorbance at 405 nm was recorded over 18 h as a reporter of the converted plasmin activity. The maximum of the first derivative of the absorbance at 405 nm (maximal enzymatic activity) as a function of the time shows a dose response in the range of concentration tested (FIG. 3 Left). While tPA shows a clear increase of the activation of plasminogen in plasmin in the absence of fibrin, OptPA has less potential to activate plasminogen in the same range of concentration (between 2-7 nM) (FIG. 3 Left), with a 84.4% (SD=4.7) reduced ability to activate plasminogen into plasminogen (FIG. 3 Right). (6 concentrations tested for rtPA, 3 for OptPA, done in duplicate; n=5); Open circle=OptPA₁₅₁; open square=rtPA; ** p<0.01; **** means p<0.0001.

FIG. 4: Comparison of coagulation and thrombolysis parameters assessed on thromboelastography test between OptPA and rtPA. A-C) Coagulation parameters including Clotting Time (CT-A), Maximal Clot Firmness (MCF-B) and Clot Formation Rate (CFR-C) for OptPA and rtPA at 0.6 μg/mL and 0.9 μg/mL; D-E) Thrombolysis parameters including Lysis Onset Time (LOT-D), Clot Lysis Rate (CLR-E) and Area Under Curve (AUC-F) for OptPA and rtPA at 0.6 μg/mL and 0.9 μg/mL. The Area under the curve takes in account the formation of the clot as well as the fibrinolysis of this clot. ns=non-significant; plain square and dark grey (left)=rtPA; Open square and/or light grey (right)=OptPA.

FIG. 5: Time course thrombolysis of 1 mL blood clot immersed in rtPA bath from 1 ng/mL to 1 μg/mL. Upper panel: the relative remaining clot weight at each time point of the experiment for all the treatments was monitored over 24 hours. The dashed line represents the sham condition (immersion in 0.9% NaCl during 24 hours). The black arrows represent the repeated exposure to 30 μg/mL in the corresponding conditions (three transient exposures of 15 min followed by immersion in 0.9% NaCl bath). The results are the mean ±standard deviation (SD) of the relative residual weights, n=7. Bottom panel: representative exposure of the blood clot to rtPA by unit of time and by unit of concentration.

FIG. 6: Time course thrombolysis of 1 mL blood clot immersed in rtPA bath from 1 μg/mL to 30 μg/mL. Upper panel: the relative remaining clot weight at each time point of the experiment for all the treatments was monitored over 24 hours. The dashed line represents the sham condition (immersion in 0.9% NaCl during 24 hours). The black arrows represent the repeated exposure to 30 μg/mL in the corresponding conditions (three transient exposures of 15 min followed by immersion in 0.9% NaCl bath). The results are the mean ±standard deviation (SD) of the relative residual weights, n=7. Bottom panel: representative exposure of the blood clot to rtPA by unit of time and by unit of concentration.

FIG. 7: Observational study of the thrombolytic effect of the poloxamer formulation candidates over 24h on 1 mL human whole blood clot. Upper panel: Overnight clotted human blood (1 mL). middle panel: Evaluation of the liquefaction of the blood clots by inverting the tube bottom-up 24 h after the addition of 100 μL of the formulation candidates on the clot surface. Bottom panel: the clots were extracted and the texture of the remaining clot and the liquified blood were compared. Only sham treated blood clot is not liquefied.

FIG. 8: 5 mL human blood haematoma lysis 9 hours after initiation of the treatment with the 3 poloxamer candidates. Relative remaining weight after treatment are represented (n=2).

FIG. 9: 5 mL human blood haematoma lysis 9 hours after initiation of the treatment with P407-rtPA and 02L-001 (P407-OptPA). Relative remaining clot weights after treatment are represented after dose escalation with P407-rtPA from 0.03 to 1.00 mg per g of solution (A, n=7) and with P407-OptPA, 02L-001, from 0.03 to 1.00 mg per g of solution (B, n=6). $, means sham different from all other groups, p<0.0001; * p<0.05; *** p<0.001; one-way ANOVA followed by Dunnett's multiple comparisons test.

FIG. 10: OptPA release from 02L-001 in a USP4 system. OptPA was quantified using a RP-HPLC method in a 0.9% NaCl dissolution medium (close loop system). Sampling times were 0 (before experimentation), 15 min, 30 min, 45 min, 60 min, 120 min, 240 min, 360 min, 480 min, 1440 min. In this experiment (n=6), 60.2% (SD=14.9) of the loaded OptPA was released during the time of the experiment and reached a maximum at t=360 min (6 h).

85% (SD=3%) of this amount were released over the first hour and the remaining 15% were released over the next hours before reaching a plateau at t=6 h.

FIG. 11: Characterization of the nanoprecipitates. A) Amidolytic activity recorded after resuspension of the nanoprecipitates obtained in hexylene glycol complemented with 4 mg/mL P188 (HG4) or 25 mg/mL P188 (HG25) or 4 mg/mL P188 and 0.05% PS20 (HG4PS); or in Tetraglycol complemented with 25 mg/mL P188 (TG25). B) Amidolytic activity recorder after resuspension of the nanoprecipitates obtained in Tetraglycol complemented with 20 mg/mL P188 (TG20). C and D) fibrinolytic activity recorded after resuspension of the nanoprecipitates obtained in Tetraglycol complemented with 20 mg/mL P188 (TG20). * means p<0.05, *** p<0.001, ****p<0.0001, ns=non-significant.

FIG. 12: Amidolytic activity recovery of tenecteplase and OptPA after nanoprecipitation. Amidolytic activity recorded after resuspension of the tenecteplase (A) and OptPA (B) nanoprecipitates obtained in hexylene glycol complemented with 4 mg/mL P188 (HG4) or 20 mg/mL P188 (HG20); or in Tetraglycol complemented 4 mg/mL P188 (TG4) or 20 mg/mL P188 (TG20).

### DETAILED DESCRIPTION OF THE INVENTION

### Plasminogen Activator Nanoparticles

The inventors surprisingly showed that sustained release of plasminogen activator in the core of haematoma allows a better thrombolytic efficacy (reduction of the clot weight) than repeated injections of the same quantity of free plasminogen activator. The inventors developed a composition comprising thermoreversible polymer to allow slow release of plasminogen activator to achieve a substantial reduction of the haematoma.

Incorporation of plasminogen activator in thermoreversible polymer is restricted due to the formation of micelles leading to the formation of the hydrogel. Trapped into the micelles, plasminogen activator is not well released and loses its activity. The solution chosen to overcome this limitation is to nanoprecipitate plasminogen activator, preferably in poloxamer in order to allow high concentration loading without protein denaturation and to favour the release of the plasminogen activator with the dissolution of the hydrogel.

In a first aspect, the present disclosure relates to a nanoparticle comprising a plasminogen activator.

As used herein, the term “nanoparticle” refers to an aggregated physical unit of solid material.

Nanoparticles are understood as particles having a median diameter d50 inferior to 1 μm. The median diameter of the nanoparticle of the invention preferably ranges from 50 to 500 nm, more preferably 50 to 200 nm, more preferably 100 to 300 nm and again more preferably is notably of about 150 nm or 200 nm.

As used herein, the terms “median diameter d50” refers to the particle diameter so that 50% of the volume of the particles population have a smaller diameter. The median diameter d50 according to the invention is determined by virtue of a particle size measurement performed on the suspensions according to the method based on light diffraction and on electronic microscopy.

Plasminogen activator is a serine protease that promotes fibrinolysis by catalysing the conversion of plasminogen to plasmin.

Plasminogen activator (PA) can be a tissue plasminogen activator (tPA) that is a serine protease secreted in the neurovascular unit by endothelial cells, neurons, and glial cells. tPA is encoded by the PLAT gene and refers to the serine protease EC 3.4.21.68.

According to the present disclosure, the plasminogen activator can be Alteplase (Activase®), Tenecteplase, Palmiteplase, Monteplase, Lanoteplase, Reteplase, Desmoteplase, Urokinase, Anisoylated purified streptokinase activator complex (APSAC; Anistreplase), Streptokinase, Pro-urokinase, Staphylokinase or the W253R/R275S tPA.

In a preferred embodiment, said W253R/R275S tPA is a mutated tPA which has a good fibrinolytic activity and which does not promote N-methyl-D-aspartate receptors (NMDAR) mediated neurotoxicity as disclosed in WO2013/034710. In a preferred embodiment the W253R/R275S tPA is specifically mutated in the Lysine Binding Site present in the Kringle 2 domain of the tPA, particularly in a LBS constitutive tryptophan. In a more preferred embodiment, said W253R/R275S tPA is a mutant plasminogen activator comprising the double mutation W253R and R275S. In a more preferred embodiment, the W253R/R275S mutated tPA, also named OptPA comprises or consists of the sequence SEQ ID NO: 1.

Nanoparticle can be obtained from a variety of method. As non-limiting examples, nanoparticles can be obtained by nanoprecipitation of the plasminogen activator or by using polymers, lipids, polysaccharides and protein. Incorporation of plasminogen activator in thermoreversible polymer may be restricted due to the formation of micelles leading to a loss of plasminogen activator activity that is furthermore not well released. The nanoprecipitation of plasminogen activator, preferably in poloxamer in order to allow high concentration loading with limited protein denaturation, favour the release of active plasminogen activator with the dissolution of the hydrogel.

In a preferred embodiment, nanoparticle is obtained by nanoprecipitation. Nanoprecipitation is a well-known method based on the reduction of the quality of the solvent in which the main constituent of nanoparticles is dissolved, for example by altering pH, salt concentration solubility conditions or addition of a non-solvent is well-known by the person skilled in the art.

In a preferred embodiment, nanoparticle is obtained by precipitation method in the presence of a poloxamer. Thus, in a preferred embodiment, said nanoparticle comprises a plasminogen activator and a poloxamer.

As used herein, the term “poloxamer” is well known in the art and refers to a non-ionic block copolymer comprising a central hydrophobic chain of polyoxypropylene flanked by hydrophilic chain of polyoxyethylene. The block copolymer can be represented by the following formula: H (C₂H₄₀)ₓ(C₃H₆o)_{z}(C₂H₄₀)_{y}OH, wherein z is an integer such that the hydrophobic base represented by (C₃H₆₀) has a molecular weight of at least 2250 Da and x or y is an integer from about 8 to 180 or higher. Poloxamers are also known by the trade name of “Pluronics” or “Synperonics” (BASF). The lengths of the polymer blocks can be customized; as a result many different poloxamers exist. They notably include poloxamines such as Tetronic® 1107 (BASF). Especially preferred poloxamers are those having a hydrophile-lipophile balance (HLB) not less than 10, preferably not less than 18, and most preferably not less than 24. Most preferred poloxamers are ones that are pharmaceutically acceptable for the intended route of administration of the plasminogen activator particles.

Preferred poloxamers are composed of a central hydrophobic chain of polyoxypropylene flanked by two hydrophilic chains of polyoxyethylene. Preferably, the poloxamer is selected from the group consisting of poloxamer 188, 338 and 237, more preferably poloxamer 188.

Composition with Thermoreversible Polymers

To promote a slow release of the nanoparticle of plasminogen activator, said nanoparticle can be resuspended in a thermoreversible polymer, in particular a thermoreversible polymer that allows the release of solubilized material over a few hours, no more than 24 hours.

The present disclosure relates to a composition comprising the nanoparticle as described above and a thermoreversible polymer.

Thermoreversible polymers as used herein refer to polymers that exhibit a drastic and discontinuous change of their physical properties with temperature. In another terms, a thermoreversible polymer is characterised by a sol-gel transition temperature under which the polymer remains fluid and above which the polymer becomes semi-solid.

In a preferred embodiment, a composition comprising a plasminogen activator nanoparticle and a thermoreversible polymer can be prepared and injected in solution at low temperatures and form a gel at body temperature to allow the slow release of plasminogen activator from the gel.

Said thermoreversible polymer is preferably poloxamer, preferably poloxamer which exhibits reversible thermal gelation such as poloxamer 407. Poloxamer 407 is particularly suitable for the use according to the invention as in aqueous solutions poloxamer 407 shows thermoreversible properties, which presents great interest in optimising drug formulation. Indeed, although at low temperature poloxamer 407 and nanoparticle of plasminogen activator are in solution, they form a gel at body temperature to allow slow release of the protein from the gel.

In a more preferred embodiment, said poloxamer 407 is at a concentration between 15 to 25% (w/v), preferably between 17% (w/v) and 23%, preferably 17% (w/v).

In another embodiment, said thermoreversible polymer can also be as non-limiting examples chitosan, cellulose, gelatin and synthetic thermoresponsive polymers like poly(N-isopropylacrylamide) (pNIPAAm).

### Therapeutic Use

Said composition comprising a plasminogen activator nanoparticle and a thermoreversible polymer is particularly suitable to reduce haematoma, in particular cerebral haematoma which occurs for example in intra-parenchymatous, intra-ventricular and subarachnoid haemorrhages or haematoma diseases.

The present disclosure also relates to the therapeutic use of nanoparticle or composition as described previously.

In particular the nanoparticle or composition as described above is used for treating thrombotic or haemorrhagic disease in a subject in need thereof.

The terms “subject” and “patient” are used interchangeably herein and refer to both human and non-human animals. As used herein, the term “subject” denotes a mammal, such as a rodent, a feline, a canine, and a primate. Preferably, a subject according to the invention is a human.

As used herein, the term “treatment”, “treat” or “treating” refers to any act intended to ameliorate the health status of patients such as therapy, prevention, prophylaxis and retardation of the disease. In certain embodiments, such term refers to the amelioration or eradication of a disease or symptoms associated with a disease. In other embodiments, this term refers to minimizing the spread or worsening of the disease resulting from the administration of one or more therapeutic agents to a subject with such a disease.

Particularly, the nanoparticle or composition described herein may be used for treating thrombotic or haemorrhagic diseases. Said thrombotic or haemorrhagic diseases include thrombotic or embolic ischemia, deep haematoma, artery or vein occlusions, cerebral haemorrhages or haematoma and ocular haemorrhages or haematoma.

In a more particular embodiment, said thrombotic or haemorrhagic disease is selected from the group consisting of: intra-parenchymatous haemorrhages or haematoma, intra-ventricular haemorrhages or haematoma, subarachnoid haemorrhages or haematoma, age related macular degeneration, central retinal occlusion, vitreous haemorrhages, deep haematoma traumatic or not and any post-surgical haematoma including cerebral haematoma or following intervention for cancer. In a more preferred embodiment, thrombotic or haemorrhagic disease is cerebral haemorrhages or haematoma such as intra-parenchymatous haemorrhages or haematoma, intra-ventricular haemorrhages or haematoma and subarachnoid haemorrhages or haematoma.

The present disclosure also relates to a method for treating thrombotic or haemorrhagic disease in a subject in need thereof comprising administering to said subject a therapeutically efficient amount of the nanoparticle or composition as described above.

As used herein, a “therapeutically effective amount” or an “effective amount” means the amount of a composition that, when administered to a subject for treating a state, disorder or condition is sufficient to effect a treatment, in particular to induce thrombus lysis and reduce the weight of the blood clot. The therapeutically effective amount will vary depending on the compound, formulation or composition, the disease and its severity and the age, weight, physical condition and responsiveness of the subject to be treated.

The administration of the nanoparticle or composition as described herein may be administered by any means known to those skilled in the art, including, without limitation, intravenously or intra-lesional administration.

In the particular case of intra-cerebral haemorrhages or haematoma including intra-parenchymatous or intra-ventricular haemorrhage or haematoma, the nanoparticle or composition described herein can be administered via intra-cerebral route.

In the particular case of subarachnoid haemorrhages or haematoma, the nanoparticle or composition described herein can be administered locally.

In the particular case of deep haematoma, the nanoparticle or composition described herein can be administered via intra-muscular or intra-articular route.

In the particular case of intramedullary spinal cord haemorrhage, the nanoparticle or composition described herein can be administered via intra-medullar route.

In the particular case of ocular haemorrhage, the nanoparticle or composition described herein can be administered via the intra-ocular route. The intra-ocular route includes intra-vitreous administration and the orbital floor route of administration.

Thus, preferably the nanoparticles or compositions may be formulated as an injectable formulation and may contains vehicles which are pharmaceutically acceptable for a formulation capable of being injected such as isotonic, sterile, saline solutions (monosodium or disodium phosphate, sodium, potassium, calcium or magnesium chloride and the like or mixtures of such salts) or dry, especially freeze-dried compositions which upon addition, depending on the case, of sterilized water or physiological saline, permit the constitution of injectable solutions.

Administration of the nanoparticle or composition as described previously to a subject in accordance with the present disclosure may exhibit beneficial effects in a dose-dependent manner. Thus, within broad limits, administration of larger quantities of the compositions is expected to achieve increased beneficial biological effects than administration of a smaller amount. Moreover, efficacy is also contemplated at dosages below the level at which toxicity is seen may be carried out in any convenient manner.

It will be appreciated that the specific dosage of the nanoparticle or composition as described above administered in any given case will be adjusted in accordance with the nanoparticle or compositions being administered, the volume of the composition that can be effectively delivered to the site of administration, the disease to be treated or inhibited, the condition of the subject, and other relevant medical factors that may modify the activity of the compositions or the response of the subject, as is well known by those skilled in the art.

For example, the specific dose of the nanoparticles or composition for a particular subject depends on age, body weight, general state of health, diet, the timing and mode of administration, the rate of excretion, medicaments used in combination and the severity of the particular disorder to which the therapy is applied. Dosages for a given patient can be determined using conventional considerations, e.g., by customary comparison of the differential activities of the compositions described herein and of a known agent, such as by means of an appropriate conventional pharmacological protocol. The compositions can be given in a single dose schedule, or in a multiple dose schedule.

The formulation of the composition as described above which allows slow release of plasminogen activator at high concentration may be advantageously given in a single dose with optionally potential rescue doses.

Suitable dosage ranges for a nanoparticle or composition as described previously may be of the order of several hundred micrograms of the agent depending on the route of administration with a range from about 0.005 to 1 mg/mL of blood/day, preferably 0.01 to 1 mg/mL of blood/day, preferably in the range from about 0.03 to 0.20 mg/mL of blood/day.

In a particular embodiment, suitable dosages for a nanoparticle or a composition as described previously may be in the range from 0.3 to 3 mg/day independently to the volume of the hematoma.

### Method of Preparing Compositions

In another aspect, the present disclosure relates to a method of preparing a composition as described above. In particular said method comprises a step of preparing nanoparticle as described above and adding said nanoparticle in a thermoreversible polymer.

Nanoparticle according to the disclosure can be obtained from a variety of method. As non-limiting examples, nanoparticles can be obtained by nanoprecipitation of the plasminogen activator or by using polymers, lipids, polysaccharides and protein. In a particular, said nanoparticle is obtained by nanoprecipitation, for example by altering pH, salt concentration, solubility conditions or addition of a non-solvent is well-known by the person skilled in the art.

In a preferred embodiment, said nanoparticle is obtained by nanoprecipitating plasminogen activator in combination with a poloxamer as described in W02009/043874. Said method comprises the step of preparing an aqueous solution comprising a plasminogen activator and a poloxamer as described previously and contacting the obtained solution with a protein precipitation reagent in a sufficient amount to precipitate plasminogen activator in combination with a poloxamer to form a plasminogen activator-poloxamer nanoparticle.

In view of improving the nanoprecipitation of plasminogen activator while protecting its enzymatic core, it is particularly preferred to use a concentration of said poloxamer in aqueous solution before forming nanoparticles between 4 mg/mL and 25 mg/mL, preferably 4 mg/mL and to use a concentration of said plasminogen activator in aqueous solution before forming nanoparticles between 0.1 mg/mL and 10 mg/mL, preferably between 1 and 4 mg/mL, more preferably 2 mg/mL.

As used herein, “protein precipitation reagent” means a reagent that allows the precipitation of the protein, in particular according to the present disclosure plasminogen activator as described above. In a preferred embodiment, said protein precipitation reagent is a solvent, more preferably a biocompatible solvent. As used herein, “biocompatible” refers to those solvents which are, within the scope of sound medical judgment, suitable for contact with the tissues of human beings and animals without excessive toxicity, irritation, allergic response, or other problem complications commensurate with a reasonable benefit/risk ratio.

Preferably, the protein precipitation solvent is glycofurol also called tetraglycol or Tetrahydrofurfuryl alcohol polyethylene glycol ether (CAS: 31692-85-0) or hexylene glycol, also called 2-methl-2,4-pentanediol (CAS: 107-41-5), preferably the protein precipitation solvent is glycofurol or hexylene glycol.

The volume of glycofurol or hexylene glycol may represent 80% to 99% of the final volume consisting of protein precipitation solvent and aqueous solution.

The precipitation yield of the protein may be further optimized by adjusting three parameters: the ratio between the volumes of the aqueous phase and protein precipitation solvent, the concentration of poloxamer, the ionic strength and the mass of protein.

The protein precipitation solvent is used in a sufficient amount to precipitate the PA as nano-sized particles. A volume ratio of protein precipitation solvent/aqueous solution ranging from 5 to 100, preferably of 5, 10, 20, more preferably 5 is generally sufficient to induce the nanoprecipitation of the PA in the presence of poloxamer. In a preferred embodiment, the ratio between the volume of aqueous phase and protein precipitation solvent is 1:5.

The formation of PA-poloxamer nanoparticles may occur in a wide range of temperature. Thus, preferably, the protein precipitation solvent is contacted with said solution comprising the PA and the poloxamer at a temperature ranging from 1 to 25° C. More preferably, it is incubated at a temperature ranging from 2 to 10° C. and most preferably of about 4° C. Indeed, it has been observed that the formation of PA-poloxamer particles is more reproducible at low temperatures.

The use of a non-ionic surfactant or salt in combination with a protein precipitation reagent may promote and/or enhance the precipitation of the protein and notably allow to reach better yields of precipitation.

Thus, in a particular embodiment, the aqueous solution contains a non-ionic surfactant, preferably selected from the group polysorbate 20, 40, 60 or 80, more preferably polysorbate 20.

In another particular embodiment, the aqueous solution contains a salt. The concentration of salt preferably ranges from 0.01 M to 3M. Preferably, the salt is a water soluble electrolyte.

Tris[hydroxymethyl]haminomethane, NaCl, KCl, (NH₄)₂SO₄ or a mixture thereof may be used. Among these, NaCl is particularly preferred.

The salt and surfactant concentration of the aqueous solution may vary in a wide range. For a given amount of plasminogen activator, at a fixed solution pH, a fixed temperature and a fixed volume ratio of aqueous solution/ protein precipitation solvent, the person ordinary skilled in the art may determine a minimal suitable salt concentration and/or surfactant by routine work, typically by adding increasing amounts of salt and/or surfactant up to observing the nanoprecipitation of the protein.

The nanoparticles obtained from the process described above may be recovered from the liquid phase by using any conventional methods.

In a preferred embodiment, the nanoparticles comprising said plasminogen activator of the present disclosure can further be added in thermoreversible polymer as described above to obtain a composition injectable at room temperature and which allows slow release of plasminogen activator in body.

In a preferred embodiment, said thermoreversible polymer is a poloxamer 407, preferably at a concentration between 15 and 25% (w/v), preferably between 17 and 23% (w/v), more preferably 17% (w/v).

### Sequence for Use in Practicing the Invention

OptPA mature protein sequence (W253R/R275S
mutations underlined)
(SEQ ID NO: 1)
SYQVICRDEKTQMIYQQHQSWLRPVLRSNRVEYCWCNSGRAQCHSVPVK
SCSEPRCFNGGTCQQALYFSDFVCQCPEGFAGKCCEIDTRATCYEDQGI
SYRGTWSTAESGAECTNWNSSALAQKPYSGRRPDAIRLGLGNHNYCRNP
DRDSKPWCYVFKAGKYSSEFCSTPACSEGNSDCYFGNGSAYRGTHSLTE
SGASCLPWNSMILIGKVYTAQNPSAQALGLGKHNYCRNPDGDAKPWCHV
LKNRRLTREYCDVPSCSTCGLRQYSQPQFSIKGGLFADIASHPWQAAIF
AKHRRSPGERFLCGGILISSCWILSAAHCFQERFPPHHLTVILGRTYRV
VPGEEEQKFEVEKYIVHKEFDDDTYDNDIALLQLKSDSSRCAQEQHLLN
RTVTDNMLCAGDTRSGGPQANLHDACQGDSGGPLVCLNDGRMTLVGIIS
WGLGCGQKDVPGVYTKVTNYLDWIRDNMRP

### EXAMPLES

### Materials and Methods

### Human Blood Samples

Human blood samples and derivate were obtained from the French Establishment for Blood

(Convention PLER-UPR/2018/041) and the studies on blood donors were declared to the French Ministry of Research (Declaration DC-2018-3154). All the blood samples were harvested in pre-citrated with 3.2% buffered sodium citrate solution tubes of 2.7 ml (BD Vacutainer®, Oakville, ON, USA). The samples were received and treated on their extraction day and stored at 4° C. before use.

### OptPA Production and tPA Variants Preparation

rtPA (alteplase) and Metalyse (tenecteplase, TNKase) were from Boehringer-Ingelheim (Germany), OptPA was from Catalent (Madison, USA). The Poloxamer 188, Poloxamer 407 and the Calcium Chloride were from Sigma-Aldrich. dPBS (Gibco™) and HEPES were from Thermofisher Scientific (Waltham, Mass., USA).

Recombinant tissue factor supplemented with synthetic phospholipid (Dade® Innovin®) from Siemens Healthcare (Munich, Germany). Spectrozyme 444 was from Biomedica Diagnostics (Montreal, Canada). S-EXTEM substrates for thrombolestography as well as the ROTEM delta instrument were from Werfen (Le Pre Saint Gervais, France).

rtPA and TNKase were resuspended as described by the manufacturer. OptPA was produced using the GPEx® technology, proprietary technology of Catalent Biologics (Madison, United States of America). Approximately 90% of purity was achieved after a sequence of 3 exchange chromatography columns to isolate the protein and conditioning in a buffer containing NaCl, PO4 and arginine. All the samples were reconditioned in HEPES 0.35M pH=7.4 before nanoformulation. OptPA was produced either from a minipool of expressing cells (mentioned OptPA in the rest of the document) or from a unique selected clone (OptPA₁₅₁). Concentration was calculated using the molar extinction coefficient (ϵ=1.69 M⁻¹.cm⁻¹).

### Activity Measurement

Four tests were used to measure the activity of the protein though the process of formulation selection for the product.

### Amidolytic Activity

Plasminogen activators were incubated in the presence of a chromogenic substrate (2 μM) (Spectrozyme 444 (methyl-D-cyclohexatyrosyl-glycyl-arginine paranitroaniline acetate)). The reaction was carried out at 37° C. in a buffer containing 300 mM NaCl, 50 mM Tris-Imidazole pH=8.4 in a final volume of 200 μL. Enzymatic activity of the plasminogen activators variants was determined by measuring the increase in absorbance of the free chromophore (pNA) generated per unit time at a wavelength of 405 nm over 30 min (initial-rate-method), in a microplate reader (BIOTEK ELx 808). Analyses were performed on 5 to 10 different concentrations in duplicate from 2 to 5 independent experiments. Medians and distributions are plotted.

### Fibrinolytic Activity

A pool of human plasma (three donors) was supplemented with 20 mM calcium chloride and with each of the tPA variants at following concentrations 1.5; 2.0 and 2.5 μg/ml. The time to clot lysis was recorded by OD measurements at 405 nm at 37° C. Analyses were performed in duplicate from five independent experiments. Results are expressed as ODmax to compare the turbidity of the fibrin clots (medians and distribution are plotted); and as the time needed to obtain 50% clot lysis to compare fibrinolysis. Medians and distribution are plotted.

### Thrombolytic Activity

For ex vivo thromboelastography assays, blood was harvested by the French Establishment for Blood. Fresh blood (300 μL per assay, used within 6 hours) was studied using rotational thromboelastography (ROTEM delta, TEM®) in the presence of rtPA and OptPA (0.6 and 0.9 μg/ml) and S-EXTEM substrate. Plasminogen activators were diluted in HEPES buffer (HEPES 10 mM pH 7.4, NaCl 150 mM). Clotting Time (CT), Maximal clot firmness (MCF) and Clot Formation Rate (CFR) were measured to characterize and quantify the modification of the coagulation step. Lysis Onset Time (LOT; time needed for a decrease in MCF by 15%), Clot Lysis Rate (CLR) and the area under curve (AUC) were measured to characterize and quantify thrombolysis. Analyses were performed on 5 to 7 independent donors. Results are given as mean and 95% confidence interval.

### Plasminogen Activation

Plasminogen activators were incubated in the presence of Glu-plasminogen (Enzyme Research Laboratories, 0.2504) and of pNAPEP 1751 (Cryopep, 0.6 mM). The reaction was carried out at 37° C. in a buffer containing 150 mM NaCl, 50 mM Tris-Imidazole pH=8.0 in a final volume of 50 μL. Enzymatic activity of the plasmin (APln), converted by the action of plasminogen activator on plasminogen (AtPA) was determined by measuring the increase in absorbance of the free chromophore (pNA) generated per unit time at a wavelength of 405 nm over 18 hours, in a microplate reader (BIOTEK ELx 808). APln is the first derivate of AtPA determined by the calculation of the slope as a function of the time (Apln=dAtPA/dt, where dAtPA 4).

Ex Vivo 1 L Human Clots Immersed in rtPA Bath

Blood was harvested by the French Establishment for Blood. Blood samples from 4 individuals were pooled for each experiment. The clot formation was induced by addition of recombinant tissue factor supplemented with synthetic phospholipid (Dade® Innovin®) and of calcium chloride solution 1M at 2% and 2.5% respectively of the final 1.00 mL clot volume. The clots were incubated at 37° C. overnight. On the day of the experiment, fresh solutions of rtPA (alteplase) were prepared. Ex vivo 1 mL clots were submitted to the following conditions in 15 ml bath:
Continuous exposure to PBS (sham treatment),
Continuous exposure to rtPA (30 μg/mL, 10 μg/mL, 1 μg/mL, 100 ng/mL, 10 ng/mL and 1 ng/mL),
Intermittent 15 min exposure to rtPA at 30 μg/mL every 4 hours followed by PBS immersions in between,
15 min exposure to rtPA at 30 μg/mL followed by a PBS immersion.

To estimate clot lysis, the clots were harvested and weighted before treatment initiation and at different time points (1 h, 3 h, 5 h, 7 h and 24 h). Results were given as mean±standard deviation (SD) of the relative residual weights from 7 independent experiments.

### Nanoformulation

PEPTIDOTS®, a proprietary nanoscale pre-formulation/formulation technology of CARLINA Technologies, provides a solution to concentrate and stabilize peptides and proteins prior to their encapsulation into drug delivery systems. The technology consists of addition of a protein precipitation solvent to an aqueous solution of a protein or a peptide, leading to the formation of stable nanoparticles (nanoprecipitates).

For the nanoprecipitation of Plasminogen Activators, Poloxamer P188 is dissolved in a solution of rtPA protein in HEPES buffer by gentle mixing at room temperature. The Poloxamer P188 is added to obtain a concentration between 4 mg/mL and 25 mg/mL in the rtPA protein solution. The rtPA protein concentration is generally between 1-3 mg/ml.

The rtPA protein dissolved in HEPES buffer including 4 mg/ml to 25 mg/mL Poloxamer 188 is nanoprecipitated by addition of a protein precipitation solvent. The protein precipitation solvent which has been used for the precipitation of the rtPA proteins is Tetraglycol (Glycofurol). Examples of other protein precipitation solvent which can be used for nanoprecipitation of proteins are mPEG400 and Hexylene Glycol.

For the nanoprecipitation of the rtPA protein, Tetraglycol is added at a solvent (i.e. protein aqueous solution) to non-solvent (i.e. Tetraglycol) ratio of 1:5. The two solutions are mixed by gentle mixing, before incubation in a refrigerator for 30 minutes. Nanoprecipitated protein is thereafter collected by centrifugation (10000 g, 30 minutes). The supernatant is discarded.

The protein pellet in the centrifugation tubes obtained after the completion of the previous step is resuspended with 17% (w/v) Poloxamer P407 solution. The protein pellet is resuspended in the P407 solution to obtain a homogenous mixture. The final concentration of rtPA protein suspended in the Poloxamer P407 solution is determined by HPLC-UV analysis.

### Ex Vivo 5 mL Human Blood Haematoma Degradation

The blood samples from the EFS were received and extracted the same day. Two blood samples from 6 individuals were received per day of experiment. All the samples received were pooled and distributed in 50 mL falcon tubes. The clot formation was induced by addition of a recombinant tissue factor supplemented with synthetic phospholipid (Dade® Innovin®) and CaCl₂ as detailed previously. The 5 mL clots were incubated at 37° C. overnight (>12 hours). Ex vivo 5 mL clots were submitted to the following conditions:
a) Evaluation of the carrier solution and its concentration to achieve easy injection for the operator (17%-P407, 20%-P407, 23%-P407),
b) Evaluation of the quantity of rtPA nanoprecipitates loaded in P407 solution to achieve clot lysis (from 0.03mg/g of gel to 1.00 mg/g)
c) Evaluation of the quantity of OptPA nanoprecipitates loaded in P407 solution to achieve clot lysis (from 0.03mg/g of gel to 1.00 mg/g)

On the day of the experiment, the tested solutions were injected in the core of the clot using 25G 0.5 mm×16mm syringes (BD microlance™ 3). Negative control clots (sham group) received an injection of 400 μl dPBS in the core of the clot. Positive control clots received 3 injections of re-solubilized 1.00 mg/mL rtPA (alteplase) at 4-hour intervals (3*133 μl=400 μl as a total), also in the core of the clot, to mimic MisTIE clinical trial protocol. To estimate clot lysis, the clots were harvested and weighted before treatment initiation and at the end of the experiment (9 h). Results were given as mean±standard deviation (SD) of the relative residual weights from two experiments for the first evaluation (a), and from 6-7 independent experiments for the two last evaluations (b) and (c).

### Statistical Analyses

Statistics tests are detailed for each result. Statistical analyses were performed using GraphPad Prism v9.1.0 and R software (version 3.5.0).

The amount of clot lysis was expressed as the relative remaining weight percentage after treatment. It corresponded to the final weight after treatment (Wfinal) divided by the initial weight before treatment (Winitial) multiplied by one hundred (Equation 1).

Relative Remaining Weight percentage=W_final/W_initial×100

### Equation 1: Calculation Used to Obtain the Relative Remaining Weight Percentage After Treatment

### Release Study

### OptPA Measurement by RP-HPLC Method

OptPA was quantified in the USP4 system using a HPLC method. Briefly, the parameters were the following: C4 column Jupiter (150×4,6 mm, 5 μm, 300 Å); mobile phase A: 0.1% TFA in Deionized Water V/V; mobile phase B: 0.1% TFA in Acetonitrile V/V and a flow rate of 1.2 mL/min. Protein was detected at 220 nm and the expected retention time was about 8.8-9.5 min.

### USP4 Dissolution Method

The development was based on the compendial flow through cell method described in European Pharmacopeia (chapter 2.9.3) as well as in the US Pharmacopeia (chapter <711>).

This method will be mentioned either as flow through cell or as USP4 which is the terminology from the US Pharmacopeia.

The dissolution system (SOTAX CE7 Smart) was configured as a closed loop system allowing the use of a fixed dissolution volume and used a piston pump (SOTAX CP7-35) with Off-line HPLC measurement.

The experiments were done by using as sample size 500 mg of O2L-001 (1 mg of protein per g of gel) in 50 mL of dissolution medium (0.9% NaCl in Buffer pH 7.4). This corresponding to a final concentration of protein (OptPA) of 10 μg/mL in the dissolution medium.

### Results

OptPA has Reduced Off-Target Activity When Compared to rtPA

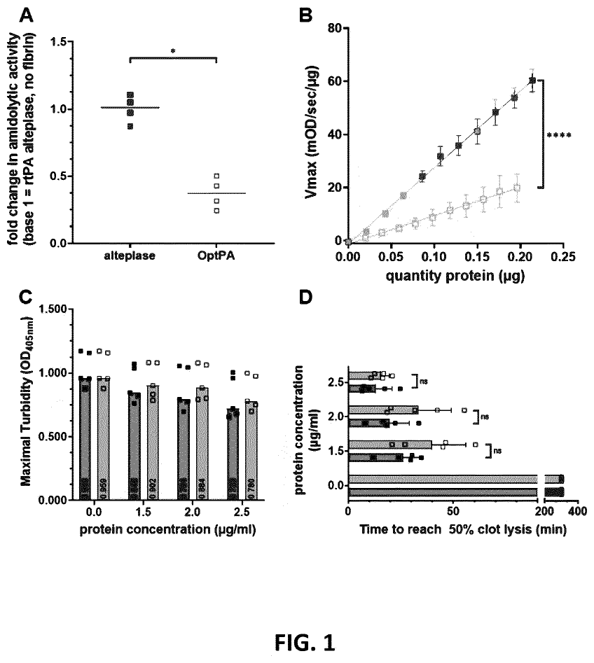

In the absence of fibrin, Optimized recombinant tPA (OptPA, W253R/R275S tPA) showed a significant 63% reduced activity to cleave Spectrozyme® rtPA, a chromogenic substrate that mimics plasminogen, when compared to rtPA (rtPA and OptPA medians were 1.01 and 0.37 respectively, n=4, p<0.05, two-tailed Mann Whitney test, FIG. 1A).

Interestingly, amidolytic activity of OptPA showed a linear dose-response lower than the one recorded with rtPA in the range of concentrations tested, from 0 to 20 μg/ml (FIG. 1B, linear regressions, slope and 95% confidence intervals are: rtPA 288.6 mOD/sec [275.0;302.2] and OptPA 107.5 mOD/sec [92.4;122.6], n=4 for each concentration, p<0.0001, F test). OptPA was then compared to rtPA in terms of fibrinolytic activity in a whole plasma clot lysis assay.

Addition of OptPA did not delay nor reduce the formation of the fibrin matrix prior fibrinolysis when compared to rtPA (ODmax medians are 0.89 for rtPA and 0.93 for OptPA, non-significant difference in a Mann-Whitney test, n=5, FIG. 1C). In the range of concentrations tested, OptPA showed non-significant difference to reach 50% lysis compared to rtPA (mean difference [95% CI] between rtPA and OptPA is 31 13.77 min [−24.00;−3.53] at 1.5 μg/ml, ns; −13.90 min [−24.14; −3.67] at 2.0 μg/ml, ns; −2.33 min [−12.57;+7.91] at 2.5 μg/ml, ns, n=5, 2-way ANOVA followed by Sidak's multiple comparisons test).

Following the identification and the isolation of a unique clone producing OptPA, the inventors characterized the enzymatic activity of the protein of interest, named OptPA₁₅₁ (produced using the GPEX® technology, Clone #151-79), on spectrozyme substrate and on pNAPEP-1751 substrate following plasminogen to plasmin activation. OptPA₁₅₁ activity was compared to alteplase (rtPA).

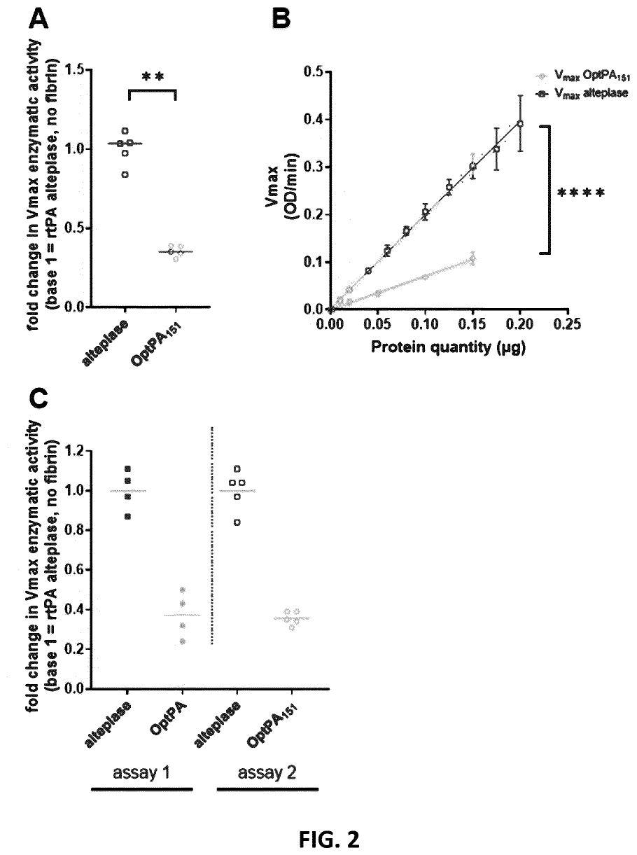

The recombinant protein obtained from the isolated clone, OptPA₁₅₁, showed the same profile as the recombinant protein obtained from the mini-pool. OptPA₁₅₁ showed a significant 68% reduced activity to cleave Spectrozyme® rtPA, when compared to alteplase (rtPA and OptPA₁₅₁ normalized medians were 1.03 and 0.35 respectively, n=5, p<0.01, two-tailed Mann Whitney test, FIG. 2A-C).

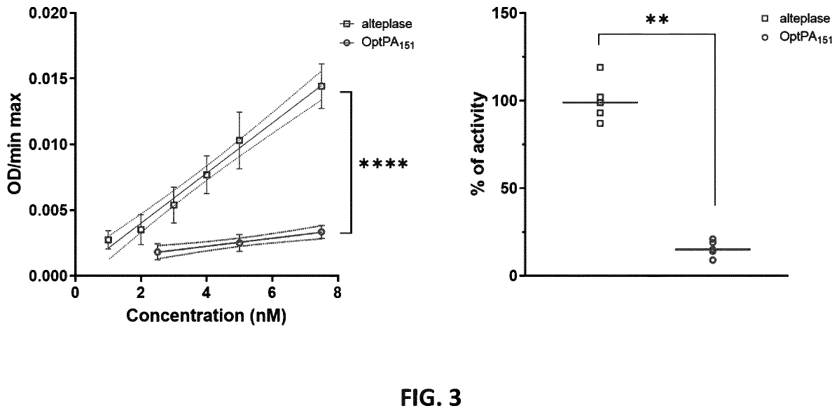

The activity of OptPA to activate plasminogen in plasmin which in turn is known to be a key player in hemorrhagic transformation, by activating gelatinases (MMPs) for example, was then recorded. Since neither tPA nor plasminogen cleaves the pNAPEP substrate (not shown here), an increase in absorbance at 405 nm reflects the formation of plasmin. The first derivative allows to calculate plasmin activity, to further plot the maximum plasmin activity as a function of the concentration. Linear regression confirmed the difference of activity between alteplase (rtPA) and OptPA₁₅₁, to trigger plasminogen to plasmin conversion, with OptPA₁₅₁ having a remarkable lower capability (slope and 95% CI are: rtPA 1.9 mOD/min/nM [1.6;2.2] and OptPA 0.3 mOD/min/nM [0.1;0.5], n=5; 6 and 3 concentrations tested for OptPA₁₅₁ and rtPA respectively, p<0.0001; F test) (FIG. 3, left). Normalization of these data showed rtPA and OptPA medians were 99% and 15% respectively, (n=5, p<0.01, two-tailed Mann Whitney test, FIG. 3, right).

Both at the level of the protein of interest, but also regarding the capacity to trigger the production of plasmin from plasminogen, OptPA showed an interesting safer profile with less enzymatic activity in the absence of fibrin vs rtPA.

The OptPA protein purified from the isolated clone, OptPA₁₅₁, shows similar performance than the protein purified from a pool of cell line, indicating robust and predictive results with the present cell line.

rtPA and OptPA Showed Similar Thrombolytic Abilities

Using thromboelastography (ROTEM delta instrument), further comparative analyses were led between rtPA and OptPA. The two thrombolytic agents were compared at 0.6 and 0.9 μg/ml on fresh human blood (harvested <6hours). These concentrations were chosen to observe the formation of the clot and the complete dissolution over a one-hour period using the EXTEM-S substrate.

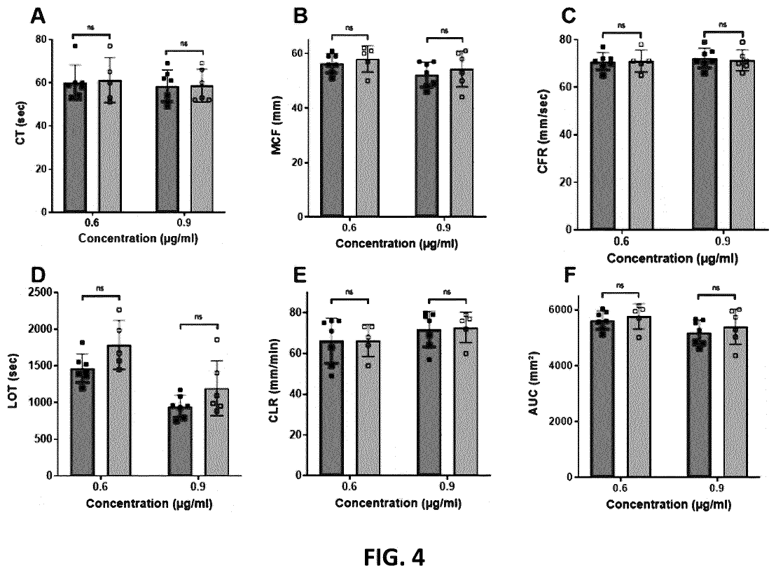

2-way ANOVA, followed by Sidak's multiple comparisons test, revealed no effect of the drug or of the concentration tested on the coagulation parameters (Clotting Time, Maximum Clot Firmness and Clot Formation Rate) (FIG. 4A-C). At the concentrations studied, rtPA and OptPA had no distinct effect on the initiation of the coagulation process (Clotting Time, the time from test start until an amplitude of 2 mm is reached) (mean difference [95% CI] between rtPA and OptPA is −0.45sec [−3.99;+3.10] at 0.6 μg/ml, ns; −0.47sec [−3.72; +2.78 ] at 0.9 μg/ml, ns, n=5-7), as well as on the clot formation rate (mean difference [95% CI] between rtPA and OptPA is −0.01° [−1.84;+1.81] at 0.6 μg/ml, ns; +1.30° [−0.37;+2.98] at 0.9 μg/ml, ns, n=5-7), as well as on the Maximum Clot Firmness (mean difference [95% CI] between rtPA and OptPA is −1.43mm [−4.66;+1.80] at 0.6 μg/ml, ns; −1.59 mm [−4.56; +1.38] at 0.9 μg/ml, ns, n=5-7, 2-way ANOVA followed by Sidak's multiple comparisons test, FIG. 4A-C).

2-way ANOVA revealed no effect of the drug on Lysis Onset Time, Clot Lysis Rate and AUC. A very significant effect of concentration was observed on these three parameters (p<0.001).

Multiple comparison did not allow to differentiate OptPA and rtPA at the concentrations tested in terms of Lysis Onset Time (FIG. 4D, mean difference [95% CI] between rtPA and OptPA is −318.9sec [−729.1;+91.31] at 0.6 μg/ml, ns; −245.8 sec [−634.8;+143.1] at 0.9 μg/ml, ns, n=5-7), Clot Lysis Rate (FIG. 4E, mean difference [95% CI] between rtPA and OptPA is −0.69° [−12.60;+11.22] at 0.6 μg/ml, ns; −1.14° [−13.05;+10.77] at 0.9 μg/ml, ns, n=5-7), and Area Under Curve (FIG. 4F, mean difference [95% CI] between rtPA and OptPA is −114.1mm² [−745;+516] at 0.6 μg/ml, ns; −199.8 mm² [−820;+421] at 0.9 μg/ml, ns, n=5-7, 2-way ANOVA followed by Sidak's multiple comparisons test).

Continuous Exposure to Low Concentration of rtpa is as Efficient as Repeated Short Exposure to High Concentration of rtpa to Liquefy a Constituted Haematoma

Haematoma resulting from haemorrhagic stroke differs from blood clot in ischemic stroke regarding the following parameters: volume of the clot, constitution of the clot, shear stress applied to the clot. Thus, as haematoma is formed outside the blood stream, the inventors wonder about the effect of lower doses of plasminogen activator with increased time of contact vs stronger and shorter exposure.

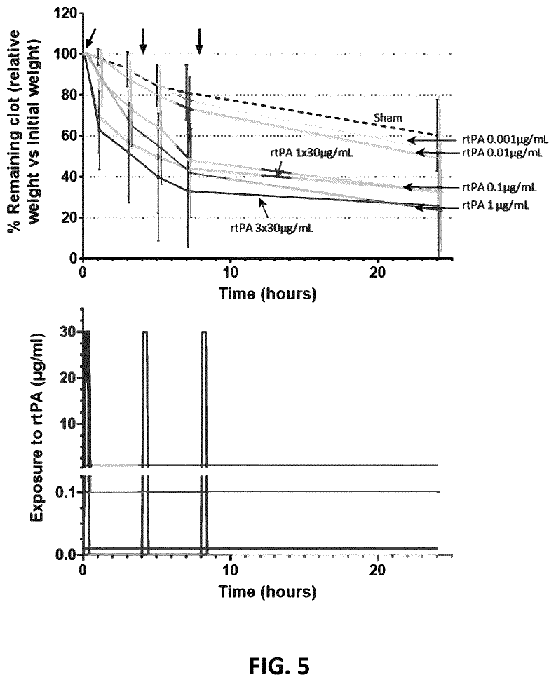

Overnight retracted 1 mL blood clots were immersed in solutions containing rtPA in order to evaluate the minimal rtPA concentration needed to achieve efficient thrombolysis. In a first set of experiments the inventors compared 24 h continuous exposure to rtPA concentration from 1 ng/mL to 1 μg/mL, to repeated 15 min exposure to 30 μg/mL (FIG. 5). 2-way ANOVA revealed a very strong effect of time (p<0.0001) and treatment (p<0.001) on blood clot lysis. Short exposure to high concentration of rtPA (15 min exposure to 30 μg/ml) led to fast clot lysis with a significant effect observed as soon as 1 hour after treatment (p<0.05), when compared to the prolonged exposure to lower concentration (ns at the four concentrations tested). Beyond the 1-hour time point, only the three consecutive exposures of 15 min to 30 μg/ml rtPA was significantly different from negative control at the different time points except at 24 h where p=0.08 (2-way ANOVA followed by Dunnett's multiple comparisons test) with a peak difference at 7 hr (mean difference [95% CI] between 3×30 μg rtPA and sham is −48.14% [−87.92;−8.36], p<0.05, n=7).

Regarding continuous exposure, no significant effect was observed one hour after the initiation of the treatment in all conditions, but a significant clot lysis was then observed until 24 h in the 1 μg/mL continuous exposure condition. At the 7 h time point, a significant blood clot lysis was achieved with both the continuous exposure at 100 ng/mL and at 1 μg/mL (mean difference [95% CI] is −33.0% [−5.6;−60.4], p<0.05; −39.3% [−7.2;−71.4], p<0.05, n=7 respectively). Interestingly, the continuous exposure to rtPA at 1 ng/mL and 10 ng/mL were never different from sham (n=7) (2-way ANOVA followed by Dunnett's multiple comparisons test).

As a conclusion of this assay, the continuous exposure at low concentration of rtPA is, at least, as efficient as repeated short exposure to high concentration of rtPA to liquefy a constituted haematoma over 24hours.

The Area Under the Curve analysis of this model shows that three consecutive exposures of 15 min at 30 μg/ml represents a global exposure of 25% higher than the continuous exposure at 1 μg/ml (30.2 arbitrary units (AU) vs 24.0 AU) and 1158% higher than the continuous exposure at 100 ng/ml (30.2 AU vs 2.4 AU) (FIG. 5—bottom). Continuous exposure of a blood clot to lower doses of plasminogen activator (here rtPA) leads to thrombolysis at lower concentrations with a similar efficiency when compared to transient concentrated exposure, with a global exposure reduced in the range of 25% to 1000%.

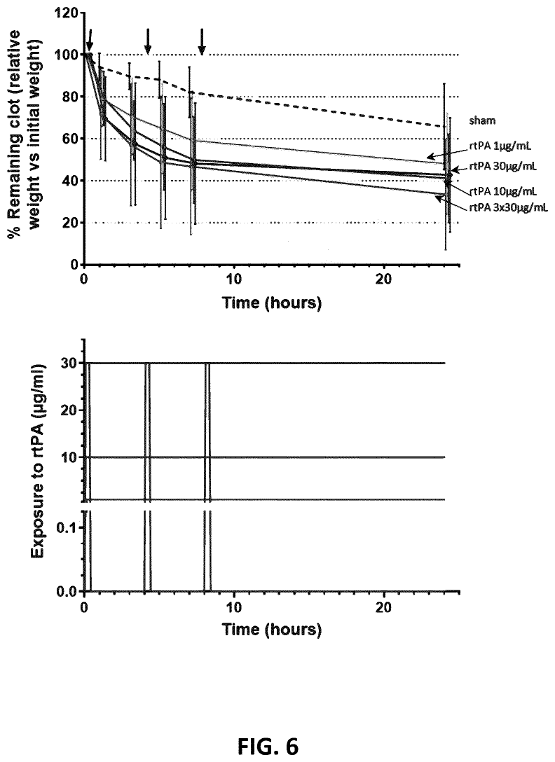

In a second set of experiments the inventors increased the concentrations of rtPA in the continuous exposure to observe whether more thrombolysis may be observable with increased continuous exposure or whether a plateau was reached. Here, the concentrations of rtPA were 1 μg/mL, 10 μg/mL and 30 μg/mL over 24 hours (FIG. 6). 2-way ANOVA revealed a very strong effect of time but an absence of effect of the rtPA concentration on blood clot lysis (respectively, p<0.0001 and p=0.08). There was no statistical difference between the continuous treatments (from 1 μg/mL to 30 μg/mL) and the successive exposure treatment (3×30 μg/mL) with rtPA (2-way ANOVA followed by Dunnett's multiple comparisons test).

To fasten and better achieve large haematoma lysis, a technology that allows the release of rtPA at concentrations above 0.1 μg/ml over a few hours, certainly more than 3 h due to enzymatic kinetics should be as efficient as multiple injections at higher concentration.

Nanoformulated rtPA (P407-rtPA) Conserves High Thrombolytic Activity

The inventors suspended rtPA nanoparticle in a solution of Poloxamer 407 which allows a sustained-release on an hour-scale. Poloxamers are synthetic polymers that exhibit thermoresponsive behavior with a finely tunable Tsol-gel (solution to gel temperature).

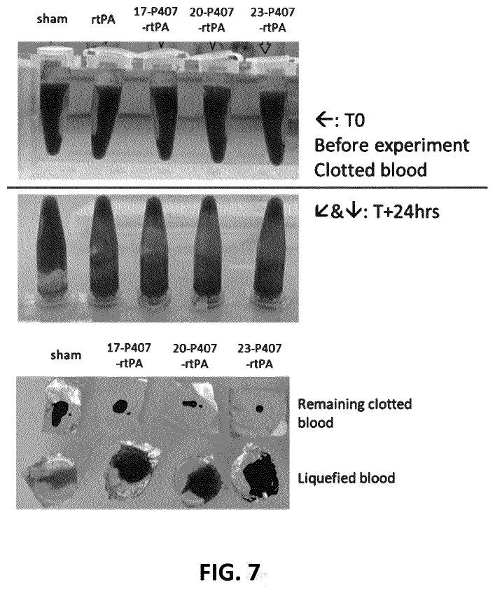

Solutions of up to 3.6 mg of rtPA per gram of gel were thus produced to validate the feasibility of the injection of such material in the core of a haematoma using a small needle (25 G 0.5mm×16 mm syringes (BD microlance™ 3)) and to check the ability of such rtPA formulation to allow blood degradation. The poloxamer candidates were produced in 17%, 20 and 23% (w/v) P407 solutions, hereafter named respectively 17-P407, 20-P407 and 23-P407 (concentrations of rtPA were respectively 3.6 mg/g; 3.6 mg/g and 3.5 mg/g) and tested in a “binomial” blood clot lysis experiment (blood clot lysed or not). 1 mL haematoma from whole human blood was prepared as described before in 1.5 mL Eppendorf® and 1000 of the poloxamer solutions were dropped on the top of the blood clot. Liquefaction was observed during 24 h and remaining clots were harvested after 24 hours. All the blood clots were liquefied by the four candidates (FIG. 7).

### 17% P407 Candidate has the Best Compatibility for Injection in the Core of the Haematoma

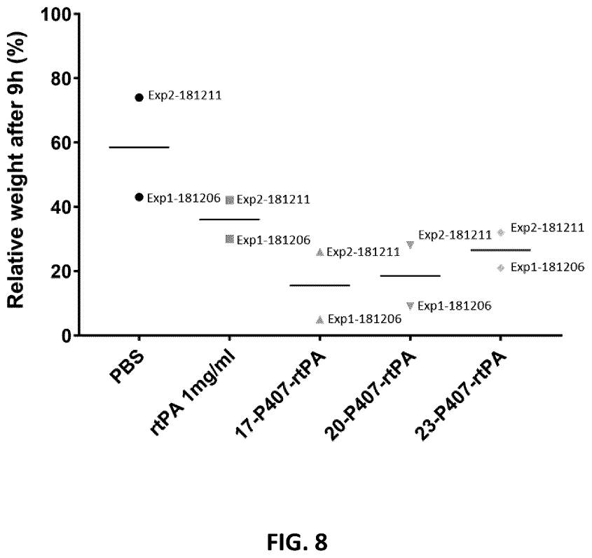

These formulations were then tested on a 5 mL whole blood haematoma with the injection of the drug in the core of the haematoma to measure haematoma liquefaction at 9 h, time point chosen based on the previous experiments (FIG. 5). All candidates achieved high clot lysis (FIG. 8). Candidates 20-P407-rtPA and 23-P407-rtPA needed a high pressure on the syringe to be injected and was less easy to handle when compared to 17-P407. Therefore, the inventors chose from this selection step the 17-P407-rtPA for an extended evaluation with a dose finding from 0.01 mg/g rtPA to 1 mg/g rtPA.

Nanoformulated Plasminogen Activator has Increased Thrombolytic Efficacy when Compared to Standard rtPA

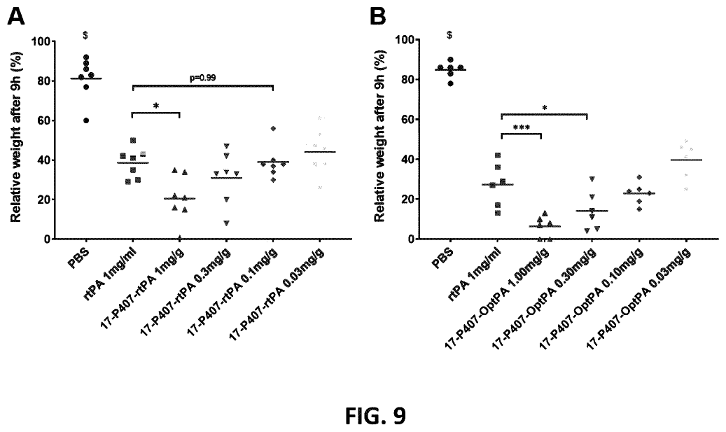

On the same model, relative weights of 5 mL haematoma were measured 9 hours after injection of either rtPA 1 mg/mL (alteplase, 400 μL injected in 3 times, 133 μL every 4 hours to mimic the MisTIE program), P407-rtPA containing 17% P407 (400 μl, concentrations administered in a single injection, concentrations ranging from 0.03 mg/g, 0.10 mg/g, 0.30 mg/g, 1.00 mg/g) (FIG. 9A).

Using a one-way ANOVA, the different candidates are compared to sham and to rtPA 1 mg/mL in terms of clot lysis. Normality of the residuals was confirmed by Kolmogorov-Smirnov test. A significant difference between sham and rtPA 1 mg/mL group (mean difference [95% CI] is +42.71% [+27.76;+57.67], p<0.0001, n=7) was observed. The post hoc analysis (Dunnett test) showed a significant difference between rtPA 1 mg/mL and 17-P407-rtPA 1 mg/g (mean difference [95% CI] is +18.00% [+3.05;+32.95], p<0.05, n=7). At the same concentration injected, 17-P407-rtPA is more efficient than rtPA not nanoformulated. Interestingly, the results observed for rtPA 1 mg/mL and 17-P407-rtPA 0.10 mg/g were similar (mean difference [95% CI] is −0.43% [−15.38;+14.53], ns, n=7) meaning that 10-fold less concentrated nanoformulated rtPA can achieve similar clot lysis than non-nanoformulated rtPA. All the rtPA groups (non-formulated and nanoformulated) were significantly different from PBS, meaning that even the lower concentration of 17-P407-rtPA was an efficient thrombolytic (p<0.0001, n=7).

These experiments were reproduced with OptPA (17-P407-OptPA, also named 02L-001) at concentrations ranging from 0.03 mg/g, 0.10 mg/g, 0.30 mg/g, 1.00 mg/g with similar results (FIG. 9B). Normality of the residuals was confirmed by Kolmogorov-Smirnov test. All the plasminogen activator groups (non-formulated rtPA and nanoformulated OptPA) were significantly different from PBS, meaning an efficient thrombolytic activity in these groups (p<0.0001, n=6). Dunnett's comparison test showed significant difference between sham and rtPA 1 mg/mL group with a very conservative effect when compared to the previous experiment (mean difference [95% CI] is +57.5% [+45.3; +69.7], p<0.0001, n=6). Further bilateral comparisons with Dunnett adjustment between rtPA 1.00mg/ml and 17-P407-OptPA 1.00mg/g showed a significant difference between these two groups (mean difference [95% CI] is +21.0% [+8.8; +33.2], p<0.001, n=6). A significant difference was also highlighted between rtPA 1.00 mg/mL and 17-P407-OptPA 0.30 mg/g (mean difference [95% CI] is +13.2% [+0.9; +25.4%], p<0.05, n=6). Here again, the results observed for rtPA 1.00mg/mL and 17-OptPA 0.1 mg/g were similar (mean difference [95% CI] is +4.5% [−7.7; +16.7], ns, n=6) meaning that 10-fold less concentrated nanoformulated OptPA can achieve similar clot lysis than non-nanoformulated rtPA.

### Release Study

Nanoformulated OptPA (O2L-001) is an innovative thrombolytic technology that allows a persistent presence of the safe thrombolytic agent OptPA in order to increase the thrombolytic activity with an intended better benefit/risk balance. The objective, in a hematoma, is to allow the release of the thrombolytic agent in less than 24 h to favor liquefaction but to avoid toxicity due to iron release, and to accelerate the reduction of the mass-effect. The release period was studied to confirm this range of release time.

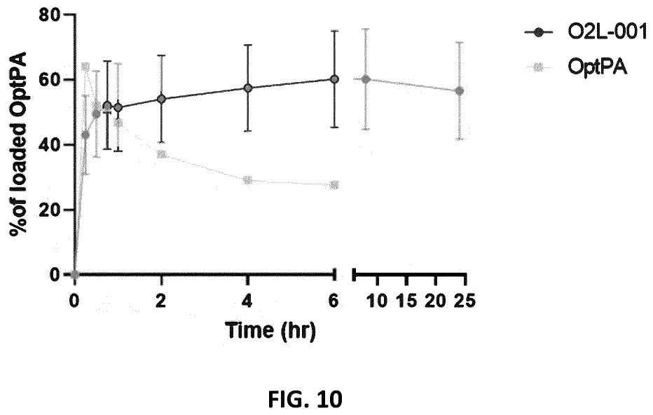

An experiment was performed with 6 repetitions of the dissolution process. Although the gelation time before dissolution analysis was not clearly identified, it is interesting to note that there are two steps in the release of the protein of interest, with an intense release time during the first 30 min (50% of the initial loaded dose released) and a slow release over the next 4-6 hours (10% of the initial loaded dose released) (FIG. 10).

These preliminary results need to be confirmed with additional experimentation, with several loaded volumes to analyze the effect of the initial volume. However, at this stage, the kinetic observed in this experiment strongly suggests that the slow release observed during the second phase could explain the distinctive effect of O2L-001 in comparison to rtPA (release as OptPA alone, in an initial burst), and the need for only low concentrations of O2L-001 to trigger thrombolysis of the 5 ml clot as observed in the previous experiment.

### High Yield Manufacturing of Plasminogen Activator Nanoprecipitates

Incorporation of protease in poloxamer thermosensible gel is restricted due to the formation of micelle as the temperature shifts and the viscosity modulus (G″) crosses the elastic modulus (G′), leading to the formation of the hydrogel. Trapped into the micelles, proteases are not well released and loss their activity. The solution chosen to overcome this limitation is to nanoprecipitate plasminogen activator before encapsulation in poloxamer to favour the release of the protease with the dissolution of the hydrogel.

Nanoprecipitation was performed as described above to allow the formation of reversible nanoprecipitates of plasminogen activators. Variables to allow high-yield nanoprecipitation are the concentration, the buffer of the protein of interest, the nature of the protein precipitation solvent, the ratio between the volume of the protein of interest and the volume of the protein precipitation solvent and the addition, polysorbate 20 (PS20) or NaCl.

The inventors analyzed up to 30 conditions of nanoprecipitation of rtPA and selected 4 conditions for which the nanoprecipitation yields were superior to 90% after HPLC analysis (Table 1).

TABLE 1
Nanoprecipitation condition tested with rtPA. Three protein precipitation solvents,
two concentrations of poloxamer 188, and addition of additives (NaCl and polysorbate
20, PS20) were tested before analysis of the nanoprecipitates obtained.

| | | | | Solvent to | | Selected for |
|---|---|---|---|---|---|---|
| Alteplase | Non-Solvent | Poloxamer | NaCl or | Non-Solvent | Precipitation | activity |
| (mg/ml) | Type | (mg/ml) | PS20 | Ratio | Yield (%) | measurements |
| 1 | Hexylene | 4 | 0 | 1:5 | 90.0 ± 13.1 | X |
| | Glycol | | | 1:10 | 73.5 ± 6.1 | |
| | | | | 1:20 | 86.8 ± 8.5 | |
| | | 25 | | 1:5 | 91.6 ± 8.2 | X |
| | | | | 1:10 | 91.7 ± 3.5 | |
| | | | | 1:20 | 93.2 ± 6.2 | |
| | | 4 | 0.05% PS20 | 1:5 | 92.0 ± 4.7 | X |
| | | | | 1:10 | 88.0 ± 3.1 | |
| | | | 0.5M NaCl | 1:10 | 82.9 ± 1.0 | |
| | | | | 1:5 | 86.4 ± 1.8 | |
| | Tetraglycol | 4 | 0 | 1:5 | 88.3 ± 8.8 | |
| | | | | 1:10 | 81.9 ± 10.3 | |
| | | | | 1:20 | 93.8 ± 9.9 | |
| | | 25 | | 1:5 | 96.1 ± 2.1 | X |
| | | | | 1:10 | 83.5 ± 6.6 | |
| | | | | 1:20 | 81.0 ± 2.0 | |
| | | 4 | 0.05% PS20 | 1:5 | 84.9 ± 2.3 | |
| | | | | 1:10 | 81.5 ± 2.1 | |
| | | | 0.5M NaCl | 1:5 | 90.6 ± 5.1 | |
| | | | | 1:10 | 86.5 ± 0.1 | |
| | mPEG550 | 4 | 0 | 1:10 | 78.8 ± 8.2 | |
| | | | | 1:20 | 94.0 ± 18.1 | |
| | | 25 | | 1:10 | 87.7 ± 3.0 | |
| | | | | 1:20 | 88.6 ± 3.7 | |
| 4 | Hexylene | 4 or 25 | 0 | 1:5, 1:10 | Aggregation, | |
| | Glycol | | | or 1:20 | discarded | |
| | Tetraglycol | | | | | |
| | mPEG550 | | | | | |

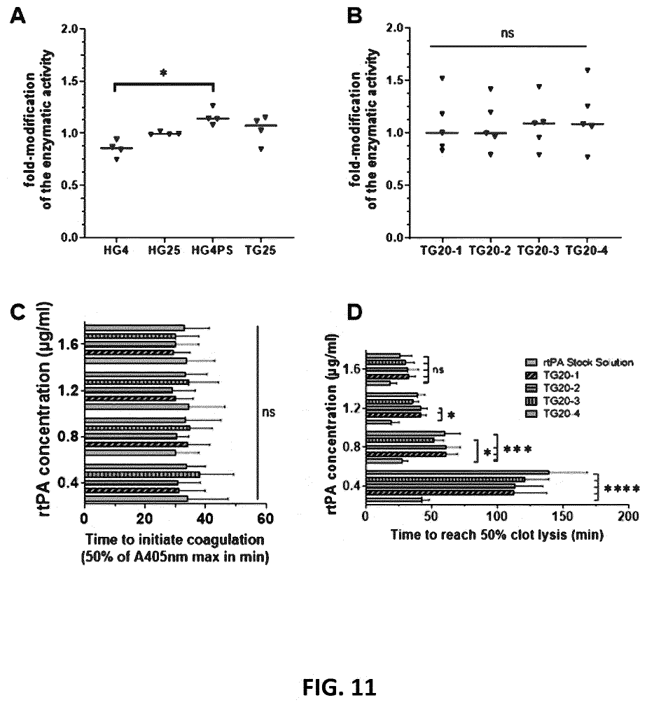

Nanoprecipitated plasminogen activators were analyzed in terms of enzymatic activity toward a chromogenic substrate mimicking plasminogen (Spectrofluor 444) after resuspension (FIG. 11). Nanoprecipitates were resuspended in a solution containing 150 mM NaCl and 10mM PO₄, pH=7.4 and keep on ice during 30 min to allow full resuspension. rtPA was sensible to the nature of the protein precipitation solvent used (tetra glycol, TG, or hexylene glycol, HG) as confirmed by a Kruskal-Wallis test (p<0.01). Addition of PS20, especially increased the enzymatic activity of the nanoprecipitates (mean rank difference −10.5, p<0.05, n=4, Dunn's multiple comparisons test) while the precipitation yields were similar (90.0%, +/−13.1 vs 92.0% +/−4.7 for HG4 vs HG4PS conditions, n=3). The other conditions tested were not statistically different (FIG. 11A).

A series of rtPA solution nanoprecipitated in tetraglycol and 20 mg/ml P188 was investigated in terms of enzymatic activity, then the fibrinolytic activity of these nanoprecipitates was measured to confirm the conservation of the thrombolytic potential of the nanoprecipitation step (FIG. 11B-D). The four samples were subjected to enzymatic activity evaluation on spectrozyme 444 rtPA substrate (n=5). There was no statistical difference between the four samples (Kruskal-Wallis test, p=0.97), with a mean enzymatic activity of 108%+/−28; 107%+/−24; 108%+/−24; 115%+/−30 in the four samples, meaning a high reproducibility of the process (FIG. 11B).

As non-nanoprecipitated rtPA, nanoprecipitated rtPA did not impair coagulation in a model of clot lysis assay in a 96-well plate, with a time to reach the inflexion point (50% of max turbidity at 405nm) comprised between 29.1 min and 34.5 min (n=5, non-significant effect of the batch or of the concentration, Two-way ANOVA, FIG. 11C). In terms of fibrinolytic activity (FIG. 11D), nanoprecipitation decreased the fibrinolytic activity of rtPA at low concentration (p<0.0001 at 0.4 μg/mL, p<0.001 at 0.8 μg/mL) with the difference being no more statistically different at the highest concentration tested (1.6 μg/mL). Indeed, at the highest concentration tested, 1.6 μg/mL, the difference in terms of time to achieve 50% clot lysis is wiped away (+19.0 min +/−4.4, from the formation of the clot vs 33.2min+/−4.6; 32.1 min+/−7.9; 30.2 min+/−6.6; 26.2min+/−8.7 in the four samples (n=5)). Interestingly, a full lysis was observed in all conditions.

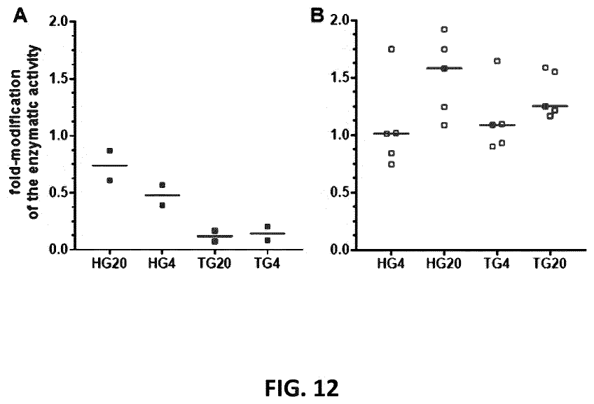

The nanoprecipitation conditions characterized with rtPA were also tested with TNKase and OptPA (FIG. 12). Tenecteplase (TNKase, a second generation of rtPA) showed low recovery in tetraglycol whatever the concentration of P188 additive (12% recovery with 20 mg/mL P188 and 14% recovery with 4 mg/mL P188, n=2 each), and a better recovery with HG (74% recovery with 20 mg/mL P188 and 48% recovery with 4 mg/mL P188, n=2 each) (FIG. 12A). OptPA, as rtPA, was efficiently nanoprecipitated in hexylene glycol and tetraglycol independently of the concentration of P188 even if a higher enzymatic activity was recorded after nanoprecipitation in hexylene glycol plus 20 mg/mL P188 (152% +/−34). In tetraglycol, nanoprecipitation led to a clear and visible pellet with the conservation of 113%+/−30 and 135.5%+/−20 enzymatic activity in the presence of 4 mg/mL and 20 mg/mL P188 respectively (n=5) (FIG. 12B). No statistical difference was observed between the groups tested (Kruskal-Wallis test, p=0,119).

1. A composition comprising a thermoreversible polymer and a nanoparticle comprising a plasminogen activator.

2. The composition of according to claim 1 wherein said nanoparticle comprises a poloxamer.

3. The composition according to claim 2 wherein said poloxamer is selected from the group consisting of: poloxamer 188, 338 and 237.

4. The composition according to claim 3 wherein said poloxamer comprises poloxamer 188.

5. The composition according to claim 1 wherein said thermoreversible polymer is a poloxamer.

6. The composition of claim 1 wherein said thermoreversible polymer is poloxamer 407.

7. The composition of claim 6 wherein said poloxamer 407 is at a concentration of from 15 to 25% (w/v).

8. The composition according to claim 1, wherein said plasminogen activator is selected from the group consisting of: rtPA, alteplase, tenecteplase, pamiteplase, monteplase, lanoteplase, reteplase, desmoteplase, urokinase, and streptokinase.

9. The composition according to claim 1, wherein said plasminogen activator is a double mutant W253R and R275S tPA.

10. (canceled)

11. A method for treating a thrombotic or haemorrhagic disease in a subject in need thereof comprising administering to said subject a therapeutically efficient amount of the composition according to claim 1.

12. The method of claim 11 wherein the thrombotic or haemorrhagic disease is selected from the group consisting of:
thrombotic or embolic ischemia, artery or vein occlusions, deep haematoma, cerebral haemorrhages or haematoma, ocular haemorrhages or haematoma, intra-ventricular haemorrhages or haematoma, subarachnoid haemorrhages or haematoma, age related macular degeneration, central retinal occlusion, vitreous haemorrhages, deep traumatic haematoma, and post-surgical haematoma including intracerebral or following intervention for cancer.

13. The method of claim 12 wherein the thrombotic or haemorrhagic disease is a cerebral haemorrhages or haematoma.

14. A method for preparing a composition comprising a thermoreversible polymer and a plasminogen activator-poloxamer nanoparticle said method comprising the steps of:
i) preparing an aqueous solution comprising a plasminogen activator and a poloxamer,
ii) contacting the aqueous solution with a protein precipitation solvent in a sufficient amount to precipitate plasminogen activator in combination with a poloxamer thereby forming plasminogen activator-poloxamer nanoparticles, and
iii) adding said plasminogen activator-poloxamer nanoparticles to a solution comprising thermoreversible polymers.

15. A nanoparticle comprising a plasminogen activator precipitated in combination with a poloxamer.

16. The composition of claim 7 wherein said poloxamer 407 is at a concentration of from 17 and 23% (w/v).

17. The composition of claim 7 wherein said poloxamer 407 is at a concentration of 17% (w/v).

18. The composition according to claim 9, wherein said double mutant W253R and R275S tPA, has the sequence set forth as SEQ ID NO: 1.

19. The method of claim 13 wherein the cerebral haemorrhage or haematoma is an intra-parenchymatous haemorrhage or haematoma, an intra-ventricular haemorrhage or haematoma or a subarachnoid haemorrhage or haematoma.

20. The nanoparticle of claim 15, wherein the poloxamer is poloxamer 188.
